(19) United States
(12) Patent Application Publication
(10) Pub. No.: US 2022/0331060 A1
(43) Pub. Date: Oct. 20, 2022

(54) Dental drill

(71) Applicant: Chia-liang Tseng, TAICHUNG CITY (TW)

(72) Inventor: Chia-liang Tseng, TAICHUNG CITY (TW)

(21) Appl. No.: 17/232,883

(22) Filed: Apr. 16, 2021

(51) Int. Cl. A61C 3/02; A61C 3/04

(52) U.S. Cl. CPC A61C 3/02; A61C 3/04

(57) ABSTRACT

A dental drill has a main body and a collecting hood. The main body has a first cutting member and a second cutting member, and both of the first cutting member and the second cutting member have a blade on two sides. A drill is formed by the first cutting member and the second cutting member with their respective bottom ends, a top end of the drill connected to a drill shank. The collecting hood has a closed end and a circular wall forming an accepting space in the collecting hood. The closed end having an engaging hole accepting the drill shank, and, a bottom inner face of the closed end has four clefts arranged in a cross shape and four pairs of the scrapers with a scraping section inbetween.

### BACKGROUND OF INVENTION

### Field of Invention

The present invention relates to a dental drill which can collect most of bone debris.

### Description of Related Art

Teeth are a part of the digestive system. They are mainly used to cut, chew, and grind food during a series of actions, so the food can be eaten smoothly. Also, the teeth can maintain facial appearance and help pronunciation and other functions, so teeth are really one of the vital organs for the human body.

However, many people have dental problems that cause the original teeth to be no longer available for use, or tooth loss due to collisions. When the dental issue occurs, it is often necessary to use artificial dental implants in the oral cavity. Dental implants are used to replace the roots of the original teeth after extraction. During the implantation process, the dentist uses a surgical instrument driller to connect a dental drill to drill holes into the alveolar bone in the patient's oral cavity, and the dental implant can be easily locked on the alveolar bone after drilling.

However, the above-mentioned conventional structure still has the following problems in practical applications: the dental drill, whether it is a twist drill or a straight flute drill, does not have the ability of properly collecting the bone debris during drilling, and the bone debris are lost due to the water flow. When the patient's alveolar bone shrinks due to periodontal disease or missing teeth for too long, it is necessary to take the patient's own bone for the bone repair procedure. The implantation of autogenous bone debris cannot be replaced by other alternative materials because of its good proliferation effect

Therefore, it is desirable to provide a dental drill to mitigate and/or obviate the aforementioned problems.

### SUMMARY OF INVENTION

An objective of present invention is to provide a dental drill, which is capable of improving the above-mention problems.

In order to achieve the above mentioned objective, a dental drill has a main body and a collecting hood. The main body has a first cutting member and a second cutting member, both of the first cutting member and the second cutting member having a blade on two sides, the first cutting member provided with a vertical jacket through a central portion, the vertical jacket accepting one side of the second cutting member passing through, such that the first cutting member and the second cutting member form four collecting grooves in a crossed arrangement and secured together; wherein a drill is formed by the first cutting member and the second cutting member with their respective bottom ends, a top end of the drill connected to a drill shank, and the drill shank has a ring groove for engaging with the collecting hood. The collecting hood has a closed end and a circular wall forming an accepting space in the collecting hood, the accepting space has an open lower end, the closed end having an engaging hole at a central position accepting the drill shank, and an edge of the engaging hole engages with the ring groove of the drill shank such that the collecting hood is assembled onto the drill shank; and wherein the circular wall covers an upper portion of the drill, a bottom inner face of the closed end has four clefts arranged in a cross shape and four pairs of the scrapers with a scraping section inbetween; each cleft is disposed between the engaging hole and the scraping section and engages with the engaging hole without protruding from the closed end, such that the clefts are expanded while the collecting hood is pushed downwardly.

Other objects, advantages, and novel features of invention will become more apparent from the following detailed description when taken in conjunction with the accompanying drawings.

### BRIEF DESCRIPTION OF DRAWINGS

FIG. 1 is a three-dimensional combination drawing of a preferred embodiment according to the present invention.

FIG. 2 is a three-dimensional exploded view of the preferred embodiment according to the present invention.

FIG. 3 is a schematic drawing showing the first cutting member and the second cutting member made by stamping technology according to the present invention.

FIG. 4 is the schematic drawing showing the first cutting member and the second cutting member after being welded and solidified according to the present invention.

FIG. 5 is a sectional view of the combination of the preferred embodiment according to the present invention.

FIG. 6 is a schematic drawing showing the bone debris being brought up during drilling according to the present invention.

FIG. 7 is a schematic drawing showing where bone debris is fully collected after drilling according to the present invention.

FIG. 8 is a schematic drawing showing where the collecting hood pushes down so that the cleft is fully expanded according to the present invention.

FIG. 9 is a schematic drawing showing the collecting hood of the present invention when the bone debris is fully scraped off after the collecting hood is pushed down according to the present invention.

FIG. 10 is a three-dimensional schematic drawing showing the collecting hood as it moving down according to the present invention.

### DETAILED DESCRIPTION OF PREFERRED EMBODIMENT

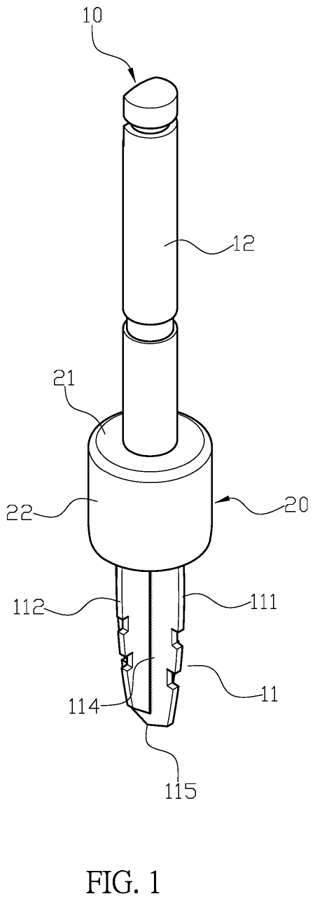

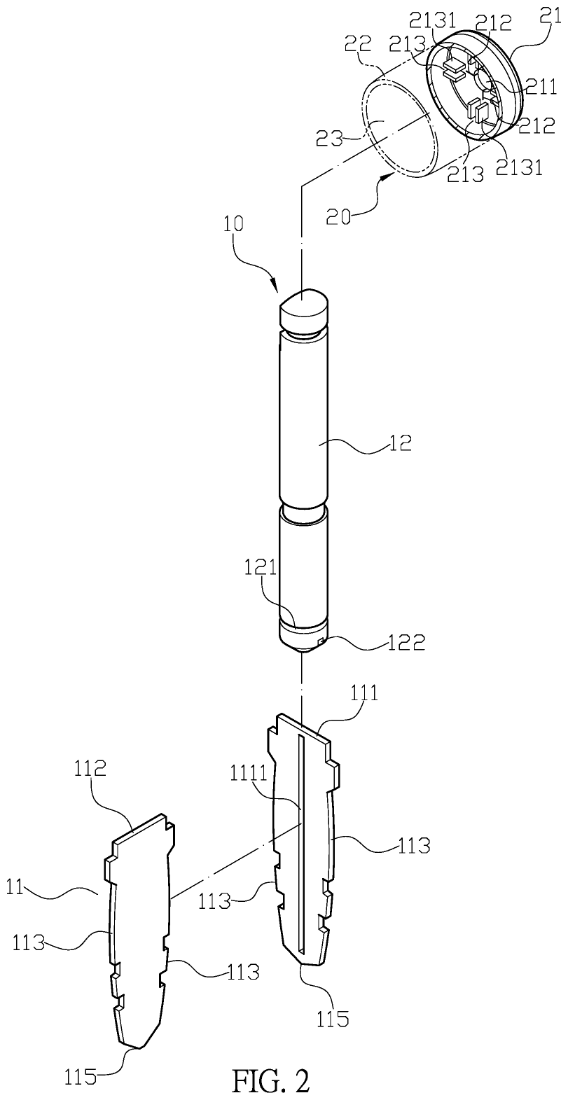

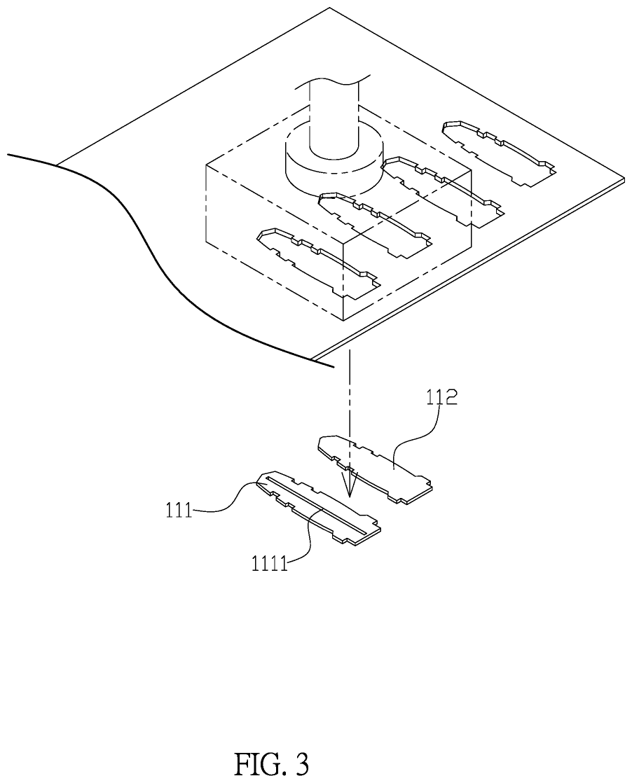

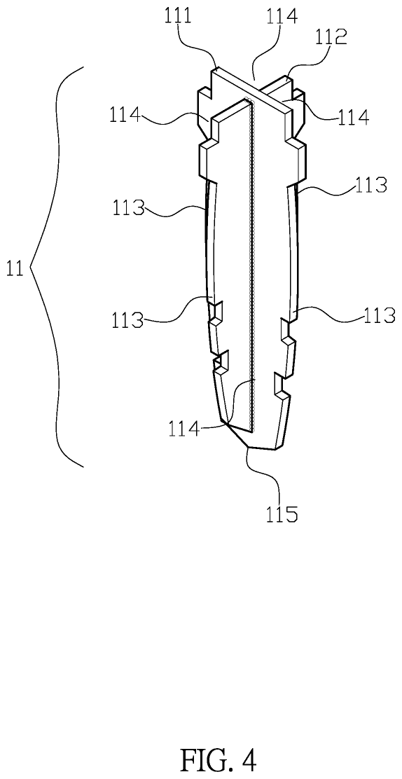

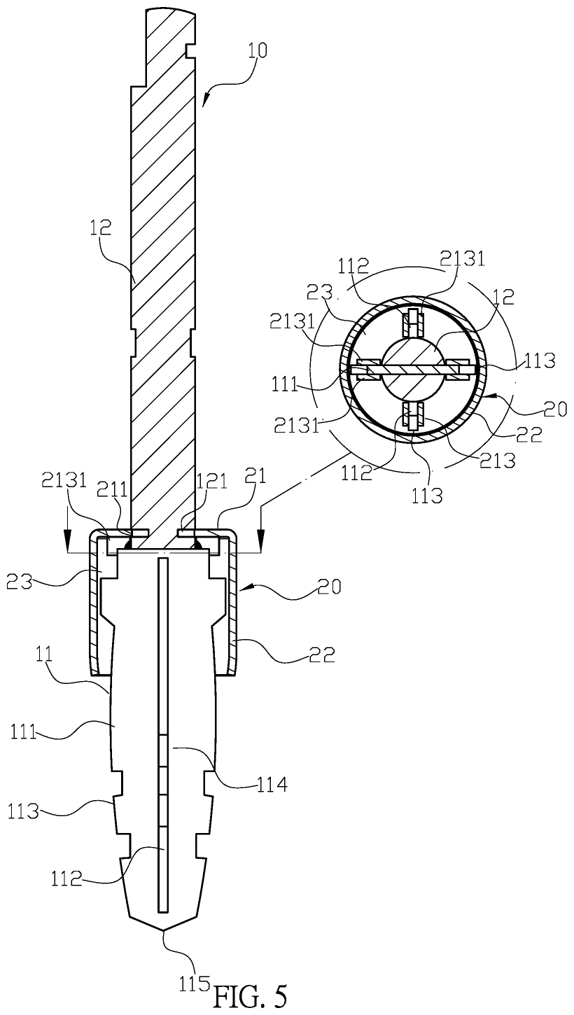

Please refer to FIGS. 1-5. A dental drill comprises a main body 10 and a collecting hood 20. The main body 10 has a first cutting member 111 and a second cutting member 112 made by stamping technology. Both of the first cutting member 111 and the second cutting member 112 have a blade 113 on two sides. The first cutting member 111 is provided with a vertical jacket 1111 through a central portion, and the vertical jacket 1111 accepts one side of the second cutting member 112 passing through the second cutting member 112 such that the first cutting member 111 and the second cutting member 112 form four collecting grooves 114 in a crossed arrangement and secured together by sodding process. A drill 115 is formed by the first cutting member 111 and the second cutting member 112 with their respective bottom ends. A top end of the drill 115 is connected to a drill shank 12, and the drill shank 12 has a ring groove 121 for engaging with the collecting hood 20. The collecting hood 20 has a closed end 21and a circular wall 22 , forming an accepting space 23 in the collecting hood 20. The accepting space 23 has an open lower end, the closed end 21 has an engaging hole 211 at a central position accepting the drill shank 12, and an edge of the engaging hole 211 engages with the ring groove 121 of the drill shank 12, such that the collecting hood 20 is assembled onto the drill shank 12. Furthermore, the circular wall 22 covers an upper portion of the drill 11. A bottom inner face of the closed end 21 has four clefts 212 arranged in a cross shape and four pairs of the scrapers 2131 with a scraping section 213 inbetween. Each cleft 212 is disposed between the engaging hole 211 and the scraping section 21 and engages with the engaging hole 211 without protruding from the closed end 21, such that the clefts 212 are expanded while the collecting hood 20 is pushed downwardly. The two scrapes 2131 of each scraping section 213 respectively stick onto the surfaces of each first cutting member 111 and the second cutting member 112, such that when the collecting hood 20 is pushed downwardly, the scrapes 2131 are capable of scraping the bone debris 30 attaching on the first cutting member 111 the second cutting member 112. Therefore, the main body 10 is able to collect the bone debris 30.

Moreover, the first cutting member 111 and the second cutting member 112 are formed by punching with a continuous die.

Furthermore, each blade 113 of the first cutting member 111 and the second cutting member 112 is a pointed cone that tapers from top to bottom.

In addition, the drill 11 and the drill shank 12 are connected by laser welding.

Also, a bottom end of the drill shank 12 has an engaging slot 122 for engaging with a top end of the first cutting member 111, and the drill 11 and the drill shank 12 are connected by laser welding.

Additionally, an end of the circular wall 22 of the collecting hood 20 is constricted inward.

Finally, the collecting hood 20 is made of medical grade plastic.

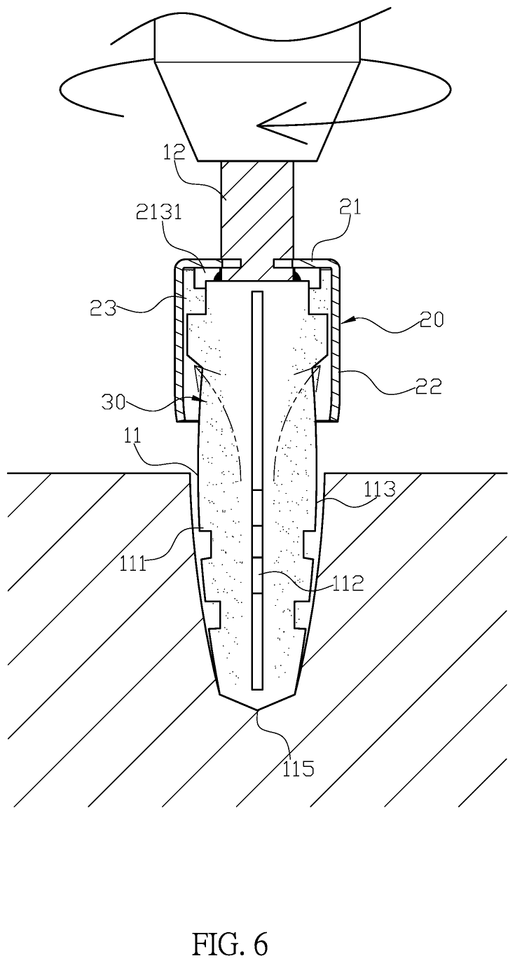

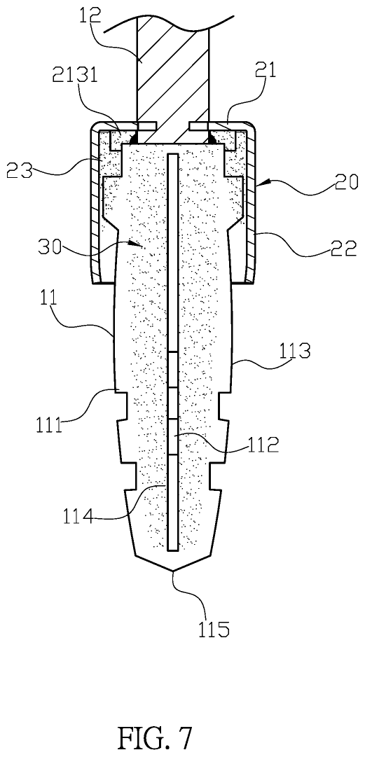

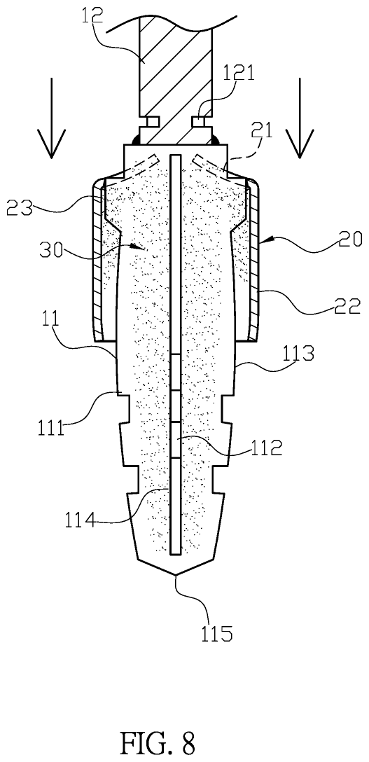

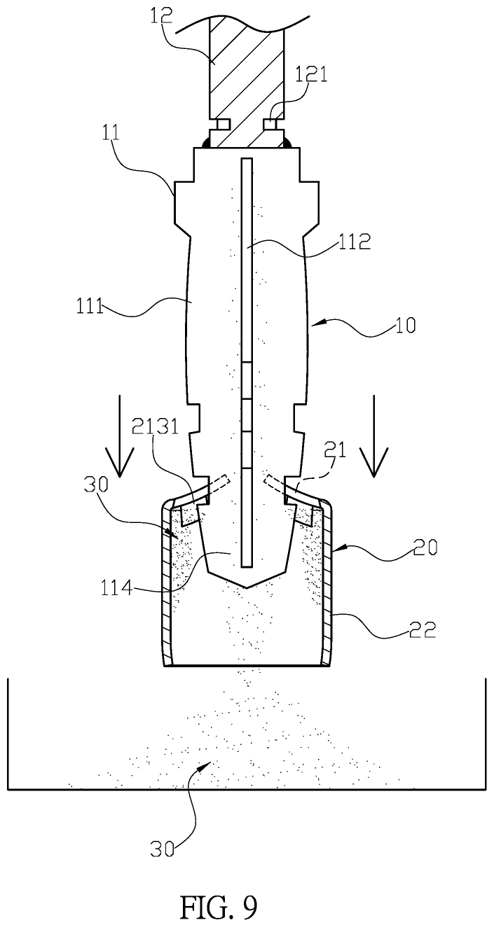

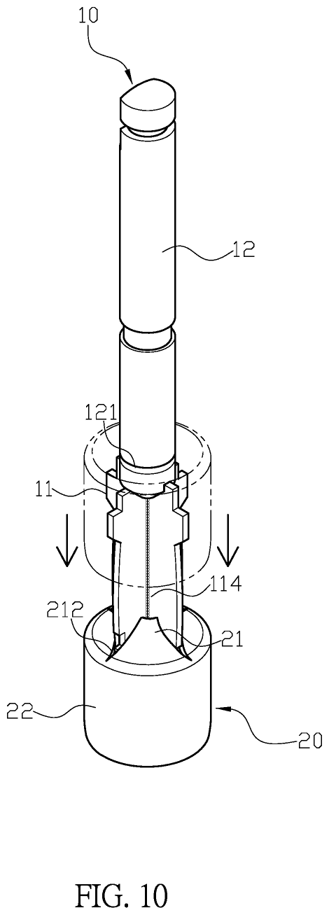

In actual use, the drill shank 12 is configured to be connected to a surgical drilling instrument for connection, so that the dentist can use the drill 115 and the blade 113 to drill the alveolar of the affected part in the patient's oral cavity. During the drilling procedure, the bone debris 30 removed from the alveolar bone is lifted up and sent into the accepting space 23 and the collecting groove 114 through the bottom open end of the collecting hood 20, as shown in FIG. 6. Water supply during drilling makes the bone debris 30 sticky, and also enables the bone debris 30 to be able to attach to the inner side of the circular wall 22 and the surface of the first cutting member 111 and the second cutting member 112. When the drilling is completed and the main body 10 is removed from the surgical instrument, as shown in FIG. 7, the collecting hood 20 is able to be pushed down, as shown in FIG. 8, and use the first cutting member 111 and the second cutting member 112 to push the inner surface of the closed end 21 of the collecting hood 20, so that the clefts 212 can be expanded open by the first cutting member 111 and the second cutting member 112. Meanwhile, the collecting hood 20 can be moved down smoothly by pushing. During the moving process, the scrapes 2131 and the expanded closed end 21 completely scraped off the bone debris 30 attaching to the surfaces of first cutting member 111 and the second cutting member 112 and contained in the collecting groove 114, as shown in FIG. 9 and FIG. 10. Therefore, the bone debris 30 can be properly and efficiently collected. Furthermore, after the main body 10 is cleaned and disinfected, it can be covered by another brand-new collecting hood 20 for reuse.

The above-mentioned dental drill has the following advantages: firstly, the collecting hood 20 is used to cover the drill 11 of the main body 10, so that the bone debris 30 generated from the drilling can be retained to the greatest extent, for bone repair procedure; after the bone debris 30 is collected, the collecting hood 20 can be moved down quickly and easily, so that the collecting of the bone debris 30 is an easy task; finally, the first cutting member 111 and the second cutting member 112 are combined by welding, which is convenient, low cost and good structural strength production.

Although the present invention has been explained in relation to its preferred embodiment, it is to be understood that many other possible modifications and variations can be made without departing from the spirit and scope of invention as hereinafter claimed.

What is claimed is:

1. A dental drill comprising a main body and a collecting hood, wherein:
the main body has a first cutting member and a second cutting member, both of the first cutting member and the second cutting member having a blade on two sides, the first cutting member provided with a vertical jacket through a central portion, the vertical jacket accepting one side of the second cutting member passing through, such that the first cutting member and the second cutting member form four collecting grooves in a crossed arrangement and secured together; wherein a drill is formed by the first cutting member and the second cutting member with their respective bottom ends, a top end of the drill connected to a drill shank, and the drill shank has a ring groove for engaging with the collecting hood; and
the collecting hood has a closed end and a circular wall forming an accepting space in the collecting hood, the accepting space has an open lower end, the closed end having an engaging hole at a central position accepting the drill shank, and an edge of the engaging hole engages with the ring groove of the drill shank such that the collecting hood is assembled onto the drill shank; and wherein the circular wall covers an upper portion of the drill, a bottom inner face of the closed end has four clefts arranged in a cross shape and four pairs of the scrapers with a scraping section inbetween; each cleft is disposed between the engaging hole and the scraping section and engages with the engaging hole without protruding from the closed end, such that the clefts are expanded while the collecting hood is pushed downwardly.

2. The dental drill as claimed in claim 1, wherein the first cutting member and the second cutting member are formed by punching with a continuous die.

3. The dental drill as claimed in claim 1, wherein each blade of the first cutting member and the second cutting member is a pointed cone that tapers from top to bottom.

4. The dental drill as claimed in claim 1, wherein the drill and the drill shank are connected by laser welding.

5. The dental drill as claimed in claim 1, wherein a bottom end of the drill shank has an engaging slot for engaging with a top end of the first cutting member, and the drill and the drill shank are connected by laser welding.

6. The dental drill as claimed in claim 1, wherein an end of the circular wall of the collecting hood is constricted inward.

7. The dental drill as claimed in claim 1, wherein the collecting hood is made of medical grade plastic.
